(19) United States
(12) Patent Application Publication
(10) Pub. No.: US 2015/0273098 A1
(43) Pub. Date: Oct. 1, 2015

(54) REMOVABLE INSERT CONTAINING SCENTED MATERIAL

(71) Applicant: Richard Martis, Melbourne Beach, FL (US)

(72) Inventor: Richard Martis, Melbourne Beach, FL (US)

(21) Appl. No.: 14/229,349

(22) Filed: Mar. 28, 2014

(51) Int. Cl. A61L 9/12

(52) U.S. Cl. CPC A61L 9/12

(57) ABSTRACT

A removable insert configured to be carried by a housing that is configured to carry a housing scented material and be removable therefrom, the removable insert comprising a lower section, a sidewall extending upwardly from the lower section, a retaining section extending outwardly from an end of the sidewall configured to interface with a surface of the housing, an insert cavity defined by the lower section and the sidewall, and an insert scented material positioned at least partially within the insert cavity. An aromatic device may include a housing, a housing scented material positioned within a housing cavity, a removable insert, and a lid. The housing scented material may have a scent that is different from a scent of the insert scented material.

### FIELD OF THE INVENTION

The present invention relates to aromatic devices, namely, removable inserts for aromatic devices.

### BACKGROUND

Aromatic devices that include foul or otherwise unpleasant scented materials are known in the art. However, the practicality of such devices is severely limited, as the intended foulness of the aromatic device necessarily makes it generally undesirable to be used beyond the initial perception of the foul scent. Such aromatic devices commonly are candles, and as such the foul-smelling scented material typically goes unused beyond an initial perception of the foul smell. This results in significant waste, as the bulk of the foul-smelling material goes unconsumed, and any material utilized in housing the foul-scented material is similarly underutilized.

Aromatic devices having generally pleasant scents have been known for millennia, but are not able to deliver the humor associated with a foul-scented material. As such, there is a need in the art for a device that enables the perception of a foul scent while reducing the waste incurred in the delivery thereof.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a removable insert configured to be carried by a housing. The housing may be configured to carry a housing scented material. Furthermore, the removable insert may be configured to be removable from the housing. The removable insert may comprise a lower section, a sidewall extending upwardly from the lower section, and a retaining section extending outwardly from an end of the sidewall configured to interface with a surface of the housing. The removable insert may further comprise an insert cavity defined by the lower section and the sidewall and an insert scented material positioned at least partially within the insert cavity.

In some embodiments, the retaining section may be generally annular and have a diameter of 7.4 centimeters. Furthermore, the lower section may be generally circular and have a diameter of 6.2 centimeters. Additionally, the sidewall may include a vertical dimension of 1.6 centimeters. A vertical offset may be defined between an upper end of the sidewall and an upper surface of the insert scented material. The vertical offset may be at least 1 centimeter. Additionally, the insert scented material may have a vertical dimension of 0.5 centimeters.

In some embodiments, at least a portion of at least one of the lower section, the sidewall and the retaining section may be formed of a thermoplastic. Furthermore, the removable insert may be devoid of a wick.

Additionally, other embodiments of the present invention are related to an aromatic device comprising a housing, a removable insert, and a lid. The housing may comprise a lower section, a sidewall extending upwardly from the lower section, and an upper section extending outwardly from the sidewall, the upper section defining an opening. A housing cavity may be defined by an internal surface of each of the lower section, the sidewall, and the upper section. Furthermore, a housing scented material may be positioned at least partially within the housing cavity.

The removable insert may be configured to be positioned at least partially within the housing cavity and be removable therefrom. The removable insert may comprise a lower section, a sidewall extending upwardly from the lower section, and a retaining section extending outwardly from an end of the one or more sidewalls. The retaining section may be configured to interface with a surface of the housing. The removable insert may further comprise an insert cavity defined by the lower section and the sidewall and an insert scented material positioned at least partially within the insert cavity.

The lid may be configured to interface and form a seal with the removable insert so as to reduce the perceptibility of the insert scented material when the lid is interfaced with the removable insert.

In some embodiments, each of the lower section and the sidewall of the removable insert may be configured such that, when the removable insert is interfaced with the upper section of the housing, the lower section of the removable insert is positioned at least partially within the housing cavity.

Furthermore, the housing scented material may define an upper surface thereof, and the removable insert may be configured so as to be carried by the upper section of the housing such that a gap is defined between the lower section of the removable insert and the upper surface of the housing scented material.

The housing scented material may be configured to be perceived as having a first scent; and the insert scented material may be configured to be perceived as having a second scent. In some embodiments, the second scent may be configured to be perceived as a different scent than the first scent.

In some embodiments, the lid may comprise an interfacing section configured to interface with at least one of an upper surface of the retaining section and an inside surface of the sidewall of the removable insert, thereby establishing a seal therebetween. The seal may reduce the perceptibility of the second scent of the insert scented material to an observer in the environment surrounding the aromatic device. The sidewall of the removable insert may define a removable insert shape and the removable insert shape may conform to the shape defined by the sidewall. Furthermore, the sidewall of the removable insert may have outer dimensions that are less than inner dimensions of the sidewall of the housing.

### BRIEF DESCRIPTION OF THE DRAWINGS

FIG. 1 is a perspective view of a removable insert according to an embodiment of the present invention.

FIG. 2 is a side elevation view of the removable insert of FIG. 1.

FIG. 3 is a perspective view of an aromatic device including the removable insert of FIG. 1.

FIG. 4 is an exploded view of the aromatic device of FIG. 3.

FIG. 5 is a side sectional view of the aromatic device of FIG. 3.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention text, as shown and described by the various figures and accompanying text, provides a removable insert. The removable insert may be configured to be carried by a housing and be removable therefrom. Additionally, the removable insert may be configured to have positioned therein scented material. Furthermore, the housing that the removable insert is carried by may itself have positioned therein a scented material that is separate from the scented material positioned in the removable insert. The scented material of the removable insert may have a scent that is different from a scent of the scented material of the housing. Furthermore, the scent of the scented material of the removable insert may be generally unpleasant, where the scent of the scented material of the housing may be generally pleasant.

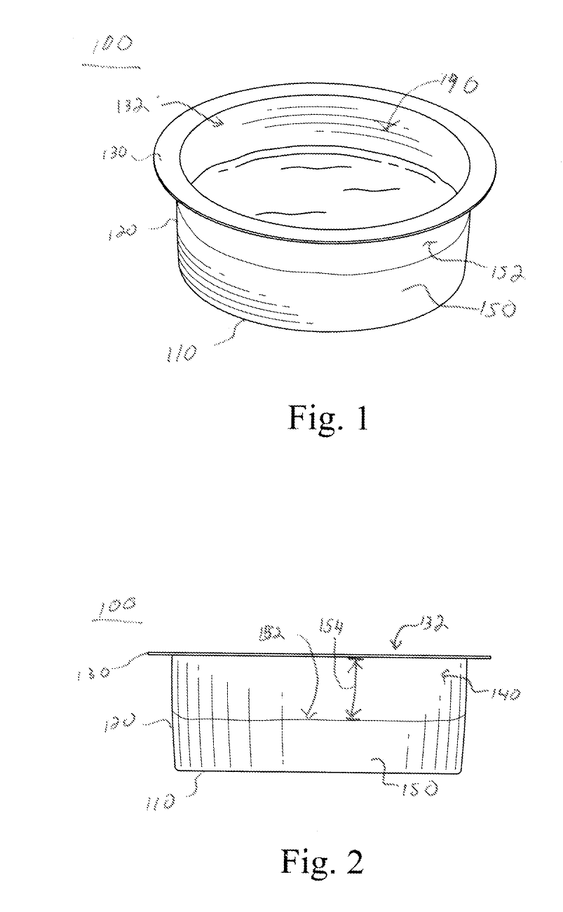

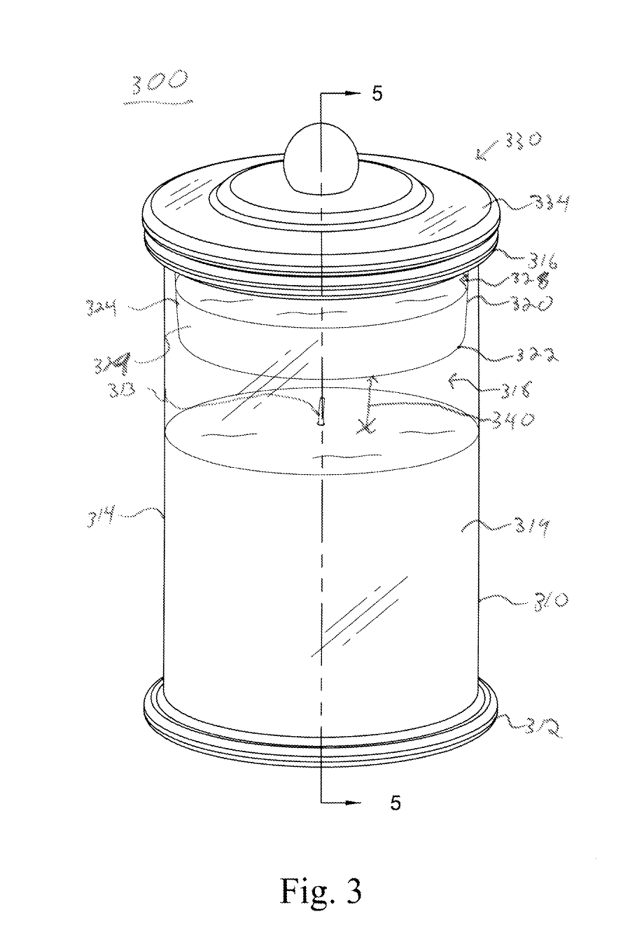

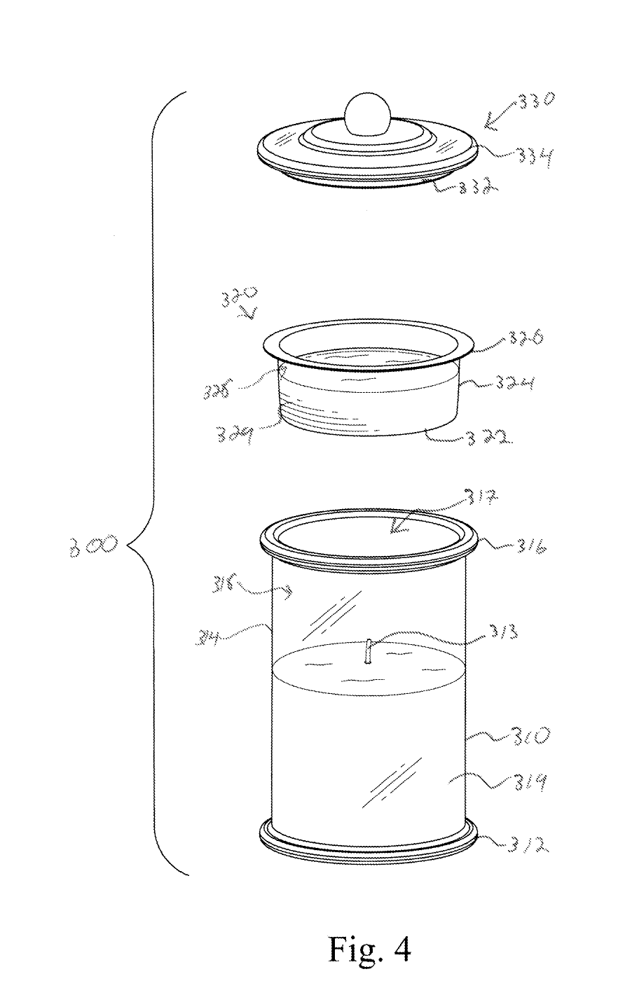

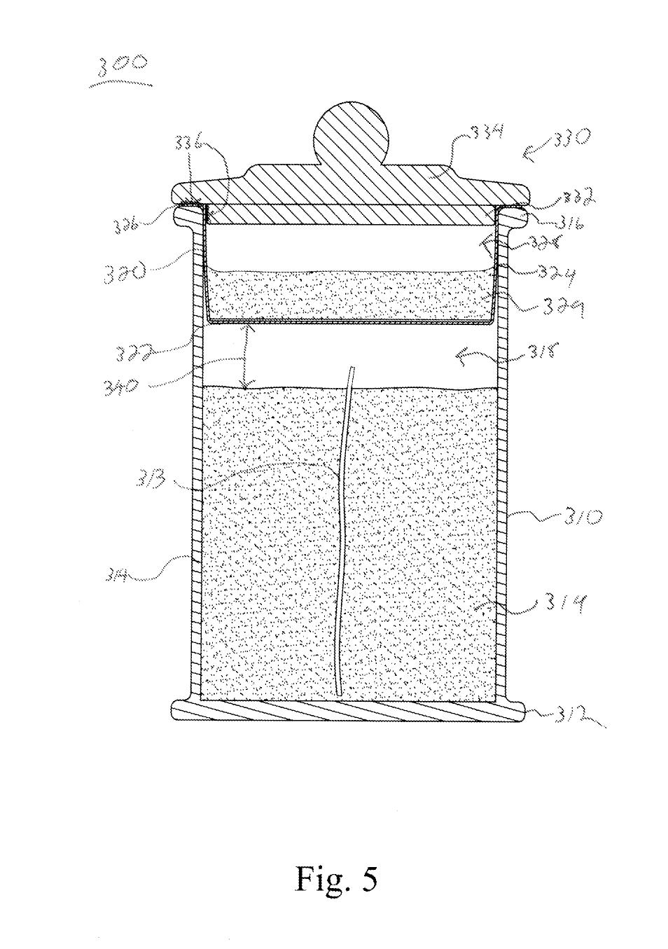

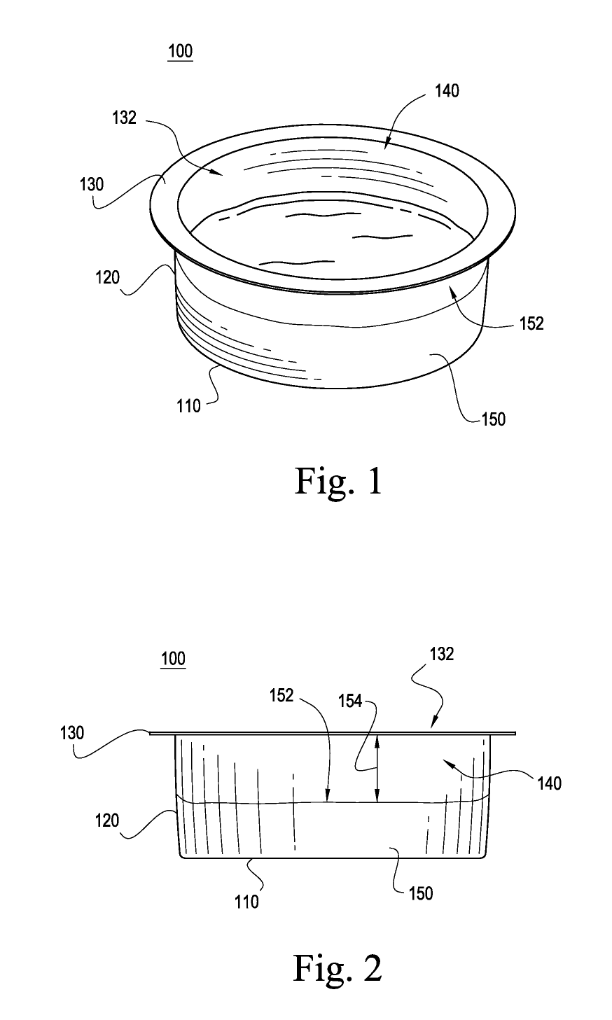

Referring now to FIGS. 1-2, a removable insert 100 according to an embodiment of the invention will now be discussed in detail. The removable insert 100 may generally be configured to define a volume within which a scented material may be positioned at least partially within, such that the scented material is carried by the removable insert 100. The removable insert 100 may include a lower section 110, a sidewall 120 extending generally upwardly from the lower section, and a retaining section 130 extending generally outwardly from an end of the sidewall 120. It is contemplated and included within the scope of the invention that any and all of the lower section 110, the sidewall 120, and the retaining section 130 may be integrally formed as a single monolithic element. Furthermore, it is contemplated and included within the scope of the invention that any of the lower section 110, the sidewall 120, and the retaining section 130 may be formed separate and apart from any of the other elements of the removable insert 100 and may be attached to adjacent elements by any means or method as is known in the art, including, but not limited to, adhesives, glues, welding, fasteners, interference fits, fusing, and the like. In the present embodiment, the sidewall 120 extends generally perpendicularly upward from an edge of the lower section 110, and the retaining section extends generally perpendicularly outward from an upper end of the sidewall 120.

Additionally, each of the lower section 110, the sidewall 120, and the retaining section 130 may be formed of any material that is suitable to be carried by a housing of an aromatic device and to permit the positioning of a scented material adjacent thereto, and to support the weight of scented material such that the scented material may be carried by the removable insert 100 without significant deformation thereof that would impede the function of the removable insert 100. Furthermore, the material may be generally chemically non-reactive with a scented material positioned adjacent thereto. Additionally, the material may be at least one of transparent and translucent. In the present embodiment, each of the lower section 110, the sidewall 120, and the retaining section 130 may be formed of a plastic, more specifically, a thermoplastic. This material is exemplary only, and any material that may accomplish the above-described features is contemplated and included within the scope of the invention.

The removable insert 100 may be configured to have a geometry defining a shape thereof, defining a removable insert shape. In some embodiments, the shape of the removable insert 100 may be defined by at least one of the lower section 110 and the sidewall 120, or both. For example, in the present embodiments, the lower section 110 is generally circular, and the sidewall 120 is generally perpendicular to the lower section 110, thereby causing the sidewall 120 to similarly be generally circular. The lower section 110 is contemplated as having any type of geometry, including, but not limited to, elliptical, ovular, triangular, rectangular, or any other polygonal shape. Moreover, whereas in the present embodiment the lower section 110 is generally flat, in some embodiments the lower section 110 may have a curvature.

Additionally, as the sidewall 120 extends from the lower section 110, the geometry of the sidewall 120 may be defined at least partially by the lower section 110. For example, in some embodiments, where the edge of the lower section 110 comprises more than one side, the sidewall 120 may have sections that correspond to each side of the edge of the lower section 110, thus causing the sidewall to have more than one side. In such embodiments, the sidewall 120 may be understood as a plurality of sidewalls. Any number of sidewalls is contemplated and included within the scope of the invention. Additionally, in some embodiments, the sidewall 120 may include more or fewer sides than the edge of the lower section 110. Furthermore, in some embodiments, an upper end of the sidewall 120 may have more or fewer sides than a lower end of the sidewall 120.

Each of at least the lower section 110 and the sidewall 120 may cooperate to define an insert cavity 140. In some embodiments, the insert cavity 140 may be understood as a volume partially bounded by at least the lower section 110 and the sidewall 120 within which a material may be positioned so as to be carried by the removable insert 100.

Additionally, as the retaining section 130 extends from the sidewall 120, the geometry of the retaining section 130 may be defined at least partially by the sidewall 120. In the present embodiment, the retaining section 130 is generally annular. However, it is contemplated and included within the scope of the invention that the retaining section 130 may have any type of geometric configuration.

The retaining section 130 may extend generally outward from the sidewall 120, thereby defining an opening 132 of the removable insert 100. The opening 132 may be configured to enable fluidic communication between the insert cavity 140 and the environment surrounding the removable insert 100. In this way, the scent of material positioned within the insert cavity 140 may be perceivable by an observer in the environment surrounding the removable insert 100.

In the present embodiment, the lower section 110 is generally circular and has a diameter of approximately 6.2 centimeters, the sidewall 120 has a vertical dimension of about 1.6 centimeters, and the retaining section 130 has an outer diameter of about 7.4 centimeters. Additionally, the retaining section 130 may have an inner diameter that is approximately equal to the diameter of the lower section 110, which, in the present embodiment, is about 6.2 centimeters.

It is contemplated and included within the scope of the invention that the dimensions of the various elements of the removable insert 100 may be selected so as to permit the removable insert 100 to be positioned at least partially within a housing of an aromatic device and carried thereby. Accordingly, any dimensions that enable such functionality within housings of aromatic devices as are known in the art are contemplated and included within the scope of the invention, and the invention is not constrained to any particular dimension or set of dimensions.

The removable insert 100 may further comprise an insert scented material 150. The insert scented material 150 may be positioned at least partially within the insert cavity 140 such that it is carried by the removable insert 100, more specifically, that interfaces with and is carried or at least partially supported by at least one of the lower section 110 and the sidewall 120.

The insert scented material 150 may define a vertical dimension thereof. In the present embodiment, the vertical dimension of the insert scented material 150 may be approximately 0.5 centimeters. In some embodiments, the vertical dimension of the insert scented material 150 may be not more than 0.5 centimeters Additionally, the insert scented material 150 may define an upper surface 152 thereof. Furthermore, a vertical offset 154 may be defined as the distance between the upper surface 152 and either of an upper end of the sidewall 120 or an inner edge of the retaining section 130. In the present embodiment, the vertical offset 154 may be about 1 centimeter. In some embodiments, the vertical offset 154 may be at least 1 centimeter.

In some embodiments, the vertical offset 154 may be configured to permit at least a portion of a lid associated with a housing of an aromatic device to be positioned within the insert cavity 140 without touching or interfacing with the upper surface 152 or any other part of the insert scented material 150. In other embodiments, the vertical offset 154 may be configured to permit at least a portion of a lid associated with a housing of an aromatic device to be positioned within the insert cavity 140 and to touch or interface with the upper surface 152.

As described hereinabove, in some embodiments, the insert scented material 150 may be configured to have a scent. In some embodiments, the scent of the insert scented material 150 may be configured to be generally unpleasant, foul, or otherwise unappealing to an observer. For this reason, as well as any other reason, it may be desirable for the removable insert 100 to not include a wick. In the present embodiment, the removable insert 100 is devoid of a wick. In other embodiments, it may be desirable for the removable insert 100 to include a wick. Such reasons for including a wick include, but are not limited to, a desire to increase the dispersion of a foul-scented material, or to increase the dispersion of a generally pleasant scented material into the environment surrounding the removable insert 100. Accordingly, in some embodiments, the removable insert 100 may include one or more wicks (not shown). Additionally, in such embodiments, the insert scented material 150 may be configured to be dispersed by combustion. Furthermore, where it may be desirable for the scent of the insert scented material 150 to be dispersed without combustion, the insert scented material 150 may be configured to increase dispersion into the environment surrounding the removable insert 100 when heated, and at least one element of the removable insert 100, including either of the lower section 110 and the sidewall 120, may be configured to have increased thermal conductivity so as to enable the heating of the insert scented material 150. In such embodiments, the removable insert 100 may be positioned in thermal communication with a heat source.

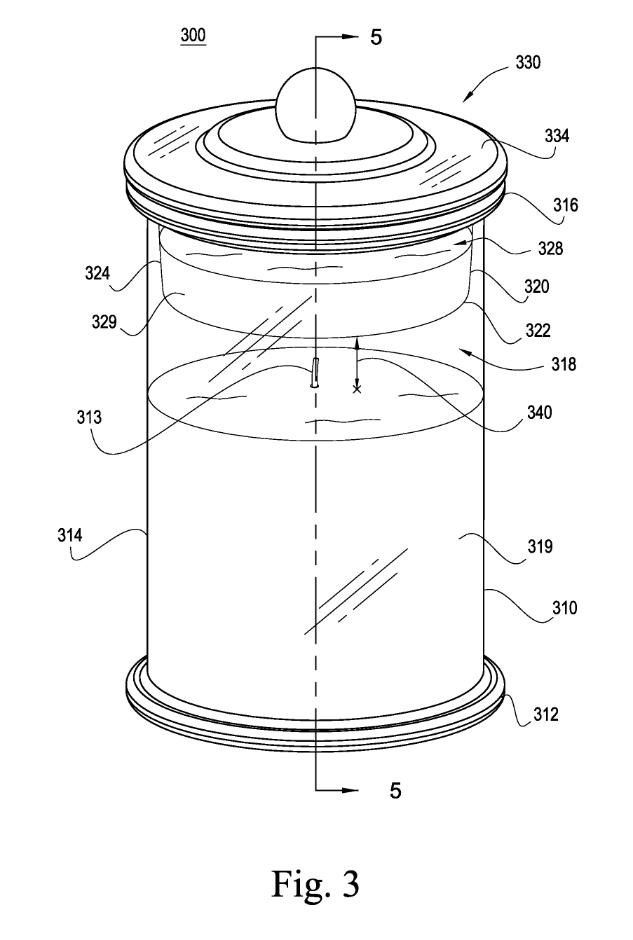

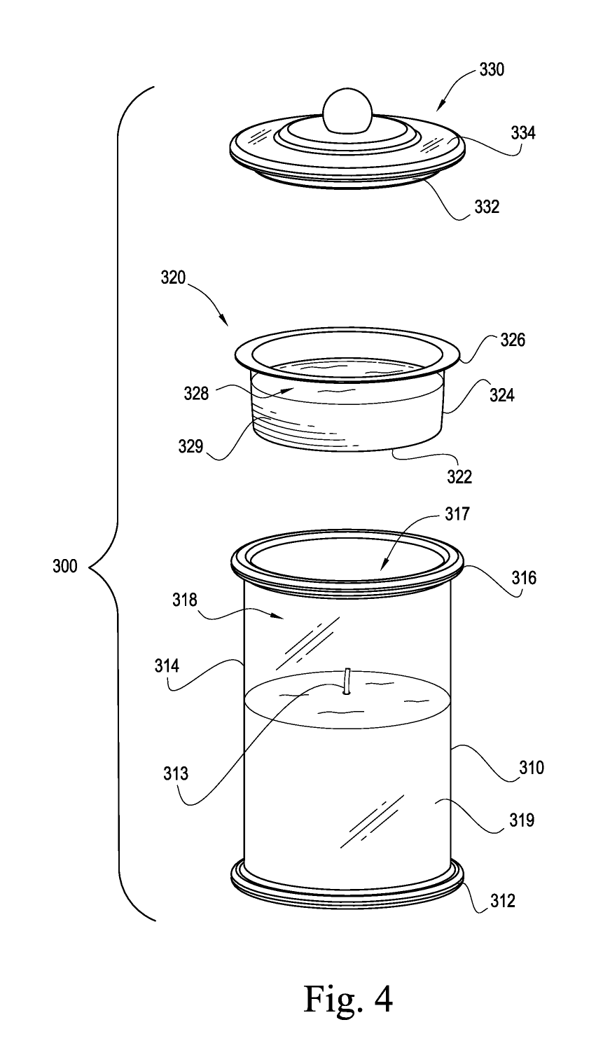

Referring now to FIGS. 3-5, an aromatic device 300 according to an embodiment of the invention will now be discussed. The aromatic device 300 may be generally understood to be a device configured to deliver scents to the environment surrounding the aromatic device 300. In the present embodiment, the aromatic device 300 may include a housing 310, a removable insert 320, and a lid 330.

The housing 310 of the aromatic device 300 will now be discussed in greater detail. The housing 310 may include a lower section 312, a sidewall 314 connected to and extending generally upwardly from the lower section 312, and an upper section 316 connected to and extending generally upwardly and/or outwardly from an upper end of the sidewall 314. Each of the lower section 312, the sidewall 314, and the upper section 316 may have a geometric configuration similar to those of the lower section 110, the sidewall 120, and the retaining section 130 of the removable insert 100 of FIGS. 1, in that they may have any geometric configuration, and have similar relationships between each of the lower section 312 and the sidewall 314, and the sidewall 314 of the upper section 316. Additionally, the geometric configurations may be generally similar to those of the various elements of the removable insert 100, or they may differ.

In the present embodiment, the lower section 312 may be generally circular and form a base of the housing 310. The sidewall 314 may extend generally upwardly from the lower section 312. Moreover, each of the lower section 312 and the sidewall 314 may define a shape thereof, and in the present embodiment, the shapes are each generally circular. Moreover, a diameter of the sidewall 314 may be generally less than a diameter of the lower section 312. Furthermore, the upper section 316 may extend generally upwardly and outwardly from the sidewall 314 so as to be generally annular. Moreover, the upper section 316 may define an opening 317. In the present embodiment, the opening 317 may have a diameter defined by an inner edge of the upper section 316. The geometric configuration of the upper section 316 of the present embodiment may generally define a lip of the housing 310.

The housing 310 may include a housing cavity 318. The housing cavity 318 may be defined at least partially by the lower section 312 and the sidewall 314 of the housing 310. Additionally, the housing cavity 318 may be configured to permit a scented material to be positioned at least partially there an and so as to permit scented material positioned therein to be carried by the housing 310, more specifically, to be carried or at least partially supported by at least one of the lower section 312 and the sidewall 314 of the housing 310.

The aromatic device 300 may further include a housing scented material 319. The housing scented material 319 may be positioned at least partially within the housing cavity 318. Additionally, the housing scented material 319 may be configured to have a first scent. Furthermore, in some embodiments, the aromatic device 300 may include a wick 313. The wick 313 may be positioned at least partially within the housing scented material 319 and may be configured to facilitate the dispersion of the housing scented material 319 by the combustion thereof. In other embodiments of the invention, the aromatic device 300 may be devoid of a wick within either of the housing scented material 319 or the housing cavity 318, or both. Additionally, in some embodiments, the aromatic device 300 may include a plurality of wicks 313.

The removable insert 320 may be substantially similar to the removable insert 100 as shown in FIGS. 1-2. More specifically, the removable insert 320 may include a lower section 322, a sidewall 324, a retaining section 326, and an insert cavity 328. Additionally, the removable insert 320 may have a geometric configuration so as to permit at least a portion thereof to be positioned at least partially within the housing cavity 318. Additionally, the removable insert 320 may have a geometric configuration so as to permit the removable insert 320 to be carried by the housing 310. More specifically, at least one of the lower section 322, the sidewall 324, and the retaining section 326 may be configured to interface with the housing 310 such that the removable insert 320 may be carried as a result of the interface. Additionally, the retaining section 326 may be configured to be generally coextensive with the upper section 316.

Additionally, the removable insert 320 may have positioned at least partially within the insert cavity 328 an insert scented material 329. The insert scented material 329 may be configured to have a second scent. In some embodiments, the second scent may be different and distinguishable from the first scent associated with the housing scented material 319. More particularly, the second scent may be generally foul or unpleasant, and the first scent may be generally pleasant. In other embodiments the first and second scents may be the same scent.

In some embodiments, when the removable insert 320 is positioned so as to be at least partially within the housing cavity 318, such a positioning may reduce the dispersion of the first scent of the housing scented material 319 into the environment surrounding the aromatic device 300, thereby reducing the perceptibility of the first scent in the environment surrounding the aromatic device 300.

Furthermore, the removable insert 320 may be configured to be removed from the housing 310 by exerting an upward force on the removable insert 320. Accordingly, any interface between the removable insert 320 and the housing 310 may permit the removal of the removable insert 320 by the exertion of relatively little force by an individual person. Furthermore, the removable insert 320 may be configured so as to be selectively re-positioned to interface with the housing 310 and be removed therefrom as many times as is desired by the individual. Accordingly, each of the removable insert 320 and the housing 310 may be formed of material that is of sufficient resiliency to withstand the exertion of forces thereon in the positioning of the removable insert 320 at least partially within the housing cavity 318 and removal therefrom without causing the deterioration of either, thereby permitting the continued carrying of the removable insert 320 by the housing 310.

In the present embodiment, the retaining section 326 may be configured to interface with the upper section 316 of the housing 310 such that the removable insert 320 may be carried by the interface between the retaining section 326 and the upper section 316. Where the removable insert 320 is positioned such that the retaining section 326 interfaces with the upper section 316, at least a portion of the removable insert 320 may be positioned within the housing cavity 318. Accordingly, in some embodiments, each of the lower section 322 and the sidewall 324 of the removable insert 320 may have outer dimension that are less than inner dimensions of the upper section 316 and the sidewall 314 of the housing 310, thereby permitting the translation of the lower section 322 and the sidewall 324 into and within the housing cavity 318. Additionally, the removable insert 320 may be configured such that, when the retaining section 326 interfaces with the upper section 316, a gap 340 is defined between an upper surface of the housing scented material 319 and a lower surface of the lower section 322 of the removable insert 320. In such a configuration, the housing scented material 319 will not interfere with the interfacing of the retaining section 326 to the upper section 316. Additionally, the gap 340 may be of a sufficient dimension such that the wick 313 may not interface with or touch any part of the removable insert 320, particularly the lower section 322.

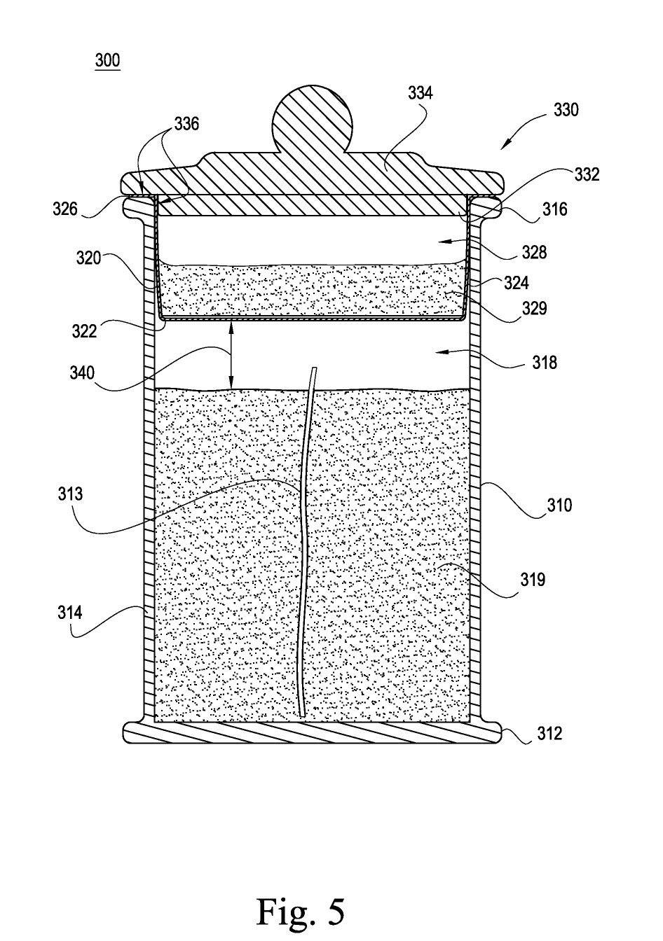

As described hereinabove, the removable insert 320 may be configured so as to permit at least a portion of the lid 330 to be positioned at least partially within the insert cavity 328. Referring now particularly to FIG. 5, it may be seen that a lower section 332 of the lid 330 may have outer dimensions that are less than inner dimensions of each of the sidewall 324 and the retaining section 326 of the removable insert 320, thereby permitting the lower section 332 of the lid 330 to be positioned within the insert cavity 328. The lid 330 may further include an upper section 334 configured to be attached to or integrally formed with the lower section 332 such that the upper section 334 is generally above the lower section 332. Moreover, the upper section 334 may extend beyond the periphery of the lower section 332, thereby permitting the upper section 334 to interface with at least one of the retaining section 326 and the upper section 316, thereby enabling the lid 330 to be carried by at least one of the housing 310 and the removable insert 320, either directly or indirectly.

Moreover, the lid 330 may be configured to interface with at least one of the elements of the removable insert 320 so as to reduce the perceptibility of the insert scented material 329. More specifically, the lid 330 may be configured to interface with at least one of the elements of the removable insert 320 so as to form a seal. The seal may be configured to prevent or substantially reduce the fluidic communication between the insert cavity 328 and the environment surrounding the aromatic device 300. Accordingly, when the lid 330 is positioned so as to interface with the removable insert 320, the second scent of the insert scented material 329 may be generally imperceptible to an observer in the environment surrounding the aromatic device 300. Furthermore, when the lid 330 is positioned so as not to interface with the removable insert 320, the second scent of the insert scented material 329 may be generally perceptible to an observer in the environment surrounding the aromatic device 300. The lid 330 may be configured to form a seal with at least one of the retaining section 326, such as an upper surface thereof, and the sidewall 324, such as an inner surface thereof, of the removable insert 320. More specifically, at least one of the lower section 332 and the upper section 334 may include an interfacing section 336 configured to interface with the removable insert 320 to form the seal. Additionally, in some embodiments, the positioning of the lid 330 at least partially within the removable insert 320 may further reduce or otherwise inhibit the dispersion of the housing scented material 319 into the environment surrounding the aromatic device 300, further reducing the perceptibility thereof.

Additionally, in some embodiments, the lid 330 may be configured to interface with the sidewall 324 so as to removably attach the removable insert 320 to the lid 330. In such embodiments, when the removable insert 320 is positioned so as to be carried by the housing 310 and the lid 330 is positioned at least partially within the insert cavity 328 such that the retaining section 336 interfaces with an element of the removable insert 320, an upward force applied to the lid 330 may cause the removable insert 320 to be lifted, thereby exposing the housing scented material 319 to the environment surrounding the aromatic device 300.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A removable insert configured to be carried by a housing that is configured to carry a housing scented material and be removable therefrom, the removable insert comprising:
a lower section;
a sidewall extending upwardly from the lower section;
a retaining section extending outwardly from an end of the sidewall configured to interface with a surface of the housing;
an insert cavity defined by the lower section and the sidewall; and
an insert scented material positioned at least partially within the insert cavity.

2. The removable insert of claim 1 wherein the retaining section is generally annular and has a diameter of 7.4 centimeters; wherein the lower section is generally circular and has a diameter of 6.2 centimeters; and wherein the sidewall has a vertical dimension of 1.6 centimeters.

3. The removable insert of claim 1 wherein a vertical offset is defined between an upper end of the sidewall and an upper surface of the insert scented material; and wherein the vertical offset is at least 1 centimeter.

4. The removable insert of claim 1 wherein the insert scented material has a vertical dimension of 0.5 centimeters.

5. The removable insert of claim 1 wherein at least a portion of at least one of the lower section, the sidewall and the retaining section is formed of a thermoplastic.

6. The removable insert of claim 1 wherein the removable insert is devoid of a wick.

7. An aromatic device comprising:
a housing comprising:
a lower section,
a sidewall extending upwardly from the lower section, and
an upper section extending outwardly from the sidewall, the upper section defining an opening,
wherein a housing cavity is defined by an internal surface of the lower section, the sidewall, and the upper section;
a housing scented material positioned at least partially within the housing cavity;
a removable insert configured to be positioned at least partially within the housing cavity and be removable therefrom, the removable insert comprising:
a lower section,
a sidewall extending upwardly from the lower section,
a retaining section extending outwardly from an end of the sidewall configured to interface with a surface of the housing, and
an insert cavity defined by the lower section and the sidewall; and
an insert scented material positioned at least partially within the insert cavity; and
a lid configured to interface with and form a seal with the removable insert so as to reduce a perceptibility of the insert scented material when the lid is interfaced with the removable insert.

8. The aromatic device according to claim 7 wherein each of the lower section and the sidewall of the removable insert are configured such that, when the removable insert is interfaced with the upper section of the housing, the lower section is positioned at least partially within the housing cavity.

9. The aromatic device according to claim 8 wherein the housing scented material defines an upper surface thereof; and wherein the removable insert is configured so as to be carried by the upper section of the housing such that a gap is defined between the lower section of the removable insert and the upper surface of the housing scented material.

10. The aromatic device according to claim 7 wherein the housing scented material is configured to be perceived as having a first scent; and wherein the insert scented material is configured to be perceived as having a second scent that is different from the first scent.

11. The aromatic device according to claim 7 wherein the lid comprises an interfacing section configured to interface with at least one of an upper surface of the retaining section and an inside surface of the sidewall of the removable insert, thereby establishing a seal therebetween.

12. The aromatic device according to claim 7 wherein the sidewall of the removable insert defines a removable insert shape.

13. The aromatic device according to claim 7 wherein the sidewall of the removable insert has outer dimensions that are less than inner dimensions of the sidewall of the housing.

14. The aromatic device according to claim 7 wherein the retaining section is generally annular and has a diameter of 7.4 centimeters; wherein the lower section of the removable insert is generally circular and has a diameter of 6.2 centimeters; and wherein the sidewall of the removable insert has a vertical dimension of 1.6 centimeters.

15. The aromatic device according to claim 7 wherein a vertical offset is defined between an upper end of the sidewall of the retaining section and an upper surface of the insert scented material; and wherein the vertical offset is at least 1 centimeter.

16. The aromatic device according to claim 7 wherein the insert scented material has a vertical dimension of not more than 0.5 centimeters.

17. The aromatic device according to claim 7 wherein the retaining section is generally coextensive with a surface of the upper section of the housing.

18. The aromatic device according to claim 7 wherein at least a portion of one of the retaining section, the sidewall, and the lower section of the removable insert is formed of a thermoplastic.

19. The aromatic device according to claim 7 wherein the removable insert is devoid of a wick.

20. An aromatic device comprising:
a housing comprising:
a lower section,
a sidewall extending upwardly from the lower section, and
an upper section extending outwardly from the sidewall, the upper section defining an opening,
wherein a housing cavity is defined by an internal surface of the lower section, the sidewall, and the upper section;
a housing scented material positioned at least partially within the housing cavity configured to be perceived as having a first scent;
a removable insert configured to be positioned at least partially within the housing and be removable therefrom, the removable insert comprising:
a lower section,
a sidewall extending upwardly from the lower section,
a retaining section extending outwardly from an end of the sidewall configured to interface with a surface of the housing, and
an insert cavity defined by the lower section and the sidewall,
wherein the sidewall of the removable insert defines a removable insert shape,
wherein the removable insert shape conforms to a shape defined by the sidewall,
wherein the sidewall of the removable insert has outer dimensions that are less than inner dimensions of the sidewall, and
wherein the removable insert is devoid of a wick;
an insert scented material positioned at least partially within the insert cavity configured to be perceived as having a second scent different from the first scent; and
a lid configured to interface with and form a seal with the removable insert so as to reduce a perceptibility of the insert scented material when the lid is interfaced with the removable insert.
